(19) United States
(12) Patent Application Publication
(10) Pub. No.: US 2010/0028311 A1
(43) Pub. Date: Feb. 4, 2010

(54) USING OF SCAFFOLD COMPRISING FIBRIN FOR DELIVERY OF STEM CELLS

(75) Inventors: Delara Motlagh, Barrington, IL (US); David L. Amrani, Glendale, WI (US); James P. Diorio, Antioch, IL (US)

Correspondence Address: MARSHALL, GERSTEIN & BORUN (BAXTER), 233 SOUTH WACKER DRIVE, 6300 SEARS TOWER, CHICAGO, IL, 60606-6357, US

(73) Assignees: BAXTER INTERNATIONAL INC., Deerfield, IL (US); BAXTER HEALTHCARE S.A., Glattpark (Opfikon) (CH)

(21) Appl. No.: 12/500,582

(22) Filed: Jul. 9, 2009

Related U.S. Application Data
(60) Provisional application No. 61/134,672, filed on Jul. 9, 2008.

(51) Int. Cl. A61K 35/12; A61P 9/10

(52) U.S. Cl. 424/93.7

(57) ABSTRACT

The invention generally relates to the field of delivery of cells to desired tissue sites, prolonged retention of the cells at the sites, and integration of cells into an area of interest for increased therapeutic effect. The invention provides, in part, compositions and methods for treating ischemia in a subject in need thereof. In some aspects, the methods of treatment comprise the administration of a fibrin scaffold or fibrin clot comprising stem cells.

This application claims benefit of U.S. Provisional Application Ser. No. 61/134,672, filed Jul. 9, 2008, which is incorporated herein by reference in its entirety.

### FIELD OF THE INVENTION

Generally, the invention relates to the field of delivery of cells to desired tissue sites, prolonged retention of the cells at the sites, and integration of cells into an area of interest for increased therapeutic effect.

### BACKGROUND OF THE INVENTION

It has long been a goal of scientists and doctors to use stem cells to treat diseases by administering these cells to sites of disease, where it is hoped that the cells will regenerate or repair the tissue. Stem cells or progenitor cells are cells that have extensive proliferation potential that differentiate into several cell lineages, and that can repopulate tissues upon transplantation. Human CD34+ stem cells are described in U.S. Pat. Nos. 4,965,204; 5,035,994; and 5,130,144. Antibody selection technology (Isolex® 300i, Baxter Healthcare Corp., Deerfield, Ill.) is used to isolate, purify, and harvest human CD34+ stem cells from a patient's blood or bone marrow (U.S. Pat. Nos. 5,536,475; 5,968,753; 6,017,719; and 6,251,295). Administration of stem cells to animals with ischemic injury is described in U.S. Pat. No. 5,980,887. A fibrin formulation using PBS to form a hydrogel is described in U.S. Pat. No. 6,965,014.

As a medical therapy, stem cells are frequently delivered using catheters or similar devices. One limitation of this approach is that there is no mechanism to retain the cells at the transplanted site or support their proliferation in situ. What is needed is a way to keep the exogenous stem cells localized at a site of injury or disease for a prolonged time for optimized therapeutic effect. The invention fills such a need by providing stem cells in a fibrin matrix for direct delivery and localization to a tissue or organ. Such a fibrin matrix provides necessary support for cells to survive over time and maintains function at the transplanted site. Delivering stem cells via this fibrin matrix has important clinical applications in treating sites of disease, injury, or ischemia in a subject in need thereof.

### SUMMARY OF THE INVENTION

The invention addresses one or more needs in the art relating to methods of delivering stem cells to a localized site of injury or disease to provide a therapeutic effect. The invention provides fibrin sealants using distinct fibrinogen/thrombin ratios that lead to enhanced and distinct cell attachment cell viability, chemokine-induced migration, and gene and protein expression in cells. Fibrin sealants create three-dimensional (3-D) structures that influence cellular functions. Various matrices were produced by altering fibrinogen/thrombin ratios using various diluents with varying initial fibrinogen concentrations to examine the resulting structure-function relationships. These alternate formulations create fibrin structures, depending on mode of delivery with CD34+ cells, that impact CD34+ cell orientation and viability in the fibrin matrix and alters fibrin-cell response properties.

In one aspect, the invention includes compositions comprising fibrin matrix and stem cells. In one aspect, the compositions of the invention comprise one or more fibrin clots and stem cells. In another aspect, the stem cells are positive for CD34 (CD34+). In yet a further aspect of the invention, the CD34+ cells are isolated by using any CD34+ selection means. In various aspects, the CD34+ cells are present in an amount from about 1,000 to about 10,000,000 cells per 1 mL of fibrin clot. In some aspects, the CD34+ cells are present in an amount from about 25,000 to about 2,000,000 cells per 1 mL of fibrin clot. In other aspects, the CD34+ cells are present in an amount from about 200,00 to about 600,000 cells per 1 mL of fibrin clot. In particular aspects, the CD34+ cells are present in an amount of about 300,000 cells per 1 mL of fibrin clot. In other aspects of the invention, the fibrin clot is Tisseel®, Tisseel® VHSD, or Floseal®. The invention includes, however, all types of fibrin and is not limited to commercially available fibrin sealants. In some aspects, the fibrin clot is in a phosphate buffer or a phosphate-buffered saline solution. In these aspects, fibrinogen and/or thrombin are prepared in the phosphate buffer. In particular aspects, Tisseel® or Tisseel® VHSD, in addition to the commercially provided buffer, also can be diluted in a phosphate buffer or a phosphate-buffered saline solution instead of the commercial buffer. In yet another aspect of the invention, the fibrin clot comprises fibrinogen at a final concentration from about 1 mg/ml to about 100 mg/ml and thrombin at a final concentration from about 1 IU/ml to about 250 IU/ml. In certain aspects, the fibrin clot comprises fibrinogen at a final concentration of about 17.5 mg/ml and thrombin at a final concentration of about 2 IU/ml. The fibrin clots of the invention are prepared in all compatible buffers. In a particular aspect, the invention includes diluting the fibrinogen and thrombin components with a phosphate buffer or a phosphate-buffered saline (PBS) solution which is compatible with CD34+ cells and allows for control of polymerization time by varying fibrinogen/thrombin ratios while obtaining a favorable fibrin structure.

In another aspect, the invention includes methods of delivering compositions comprising fibrin matrix and stem cells to a subject. In one aspect, the invention includes methods for treating a localized site of injury or disease in a subject in need thereof, the method comprising the step of delivering a composition comprising a fibrin clot and stem cells to the site of injury or disease in an amount effective for treating the injury or disease. In another aspect, the invention includes methods of enhancing vascularization to a localized site of injury or disease in a subject in need thereof, the method comprising the step of delivering a composition comprising a fibrin clot and stem cells to the site of injury or disease in an amount effective for enhancing vascularization. In yet another aspect, the invention includes methods of treating ischemia in a subject, comprising the step of delivering a composition comprising a fibrin matrix and stem cells to a site of ischemia in an amount effective to treat ischemia.

In aspects of the invention, the fibrin clot or fibrin matrix comprising stem cells is used in treating ischemia or for tissue regeneration after tissue damage or loss resulting from disease or injury. For example, tissue damage due to ischemia due to blood flow loss, lacerations, extremes of temperature, trauma, or metabolic or genetic disease, is one of many various conditions or diseases which can benefit from treatment with stem cells in a fibrin scaffold. In other aspects, the fibrin clot comprising stem cells is used in treating cardiovascular disease, diabetes, autoimmune diseases, stroke, brain and/or spinal cord injury, burn injury, bone defects, renal ischemia, and macular degeneration. In still another aspect, the fibrin clot comprising stem cells is used to treat an ischemic or a cirrhotic liver.

In certain aspects, the fibrin clot or fibrin matrix comprising stem cells of the invention is used to treat critical limb ischemia (CLI) and any of the pathophysiological processes associated with CLI, including advanced atherosclerosis, thromboembolism or atheroembolism, in situ thrombosis, and the arteritides, such as thromboangiitis obliterans (also known as TAO or Buerger disease).

The invention also includes kits for preparing a fibrin matrix comprising stem cells, wherein the kit comprises a first vial or first storage container comprising fibrinogen; a second vial or second storage container comprising thrombin; and a third vial or third storage container comprising stem cells, wherein the kit further optionally contains a phosphate buffer and instructions for use thereof.

In various aspects, the invention includes uses of a composition comprising fibrin matrix and stem cells for the manufacture of one or more medicaments. In one aspect, the invention includes the use of a composition comprising a fibrin clot and stem cells for the manufacture of a medicament to treat a localized site of injury or disease. In a further aspect, the invention includes the use of a composition comprising a fibrin clot and stem cells for the manufacture of a medicament for enhancing vascularization to a localized site of injury or disease. In still another aspect, the invention includes the use of a composition comprising a fibrin matrix and stem cells for the manufacture of a medicament for treating ischemia.

Other features and advantages of the invention will become apparent from the following detailed description. It should be understood, however, that the detailed description and the specific examples, while indicating specific embodiments of the invention, are given by way of illustration only, because various changes and modifications within the spirit and scope of the invention will become apparent to those skilled in the art from this detailed description.

### BRIEF DESCRIPTION OF THE DRAWING

A further illustration of the invention is given with reference to the accompanying drawings, which are set out below in FIGS. 1-6.

FIG. 1 shows scanning electron micrographs (SEM) of a CD34+ stem cell (see arrows) in various concentrations of fibrin matrix. FIGS. 1 (A-C) show SEMs of cells in fibrin matrix with fibrinogen and thrombin diluted in PBS: (A) 17.5 mg/ml fibrinogen and 2 U/ml thrombin; (B) 35 mg/ml fibrinogen and 2 U/ml thrombin; and (C) 50 mg/ml fibrinogen and 2 U/ml thrombin, all diluted using PBS. FIGS. 1 (D-F) show SEMs of fibrin matrix without cells with fibrinogen and thrombin diluted in PBS: (D) 17.5 mg/ml fibrinogen and 2 U/ml thrombin; (E) 35 mg/ml fibrinogen and 2 U/ml thrombin; and (F) 50 mg/ml fibrinogen and 2 U/ml thrombin, all diluted using PBS.

FIG. 2 shows polymerization curves generated for fibrin clots formed with (1) 17.5 mg/ml fibrinogen and 2 U/ml thrombin and (2) 50 mg/ml fibrinogen and 2 U/ml thrombin.

FIG. 3 shows cell viability over 8 days in fibrin formulations of 17.5/2 (17.5 mg/ml fibrinogen and 2 U/ml thrombin), 35/2 (35 mg/ml fibrinogen and 2 U/ml thrombin), and 50/2 (50 mg/ml fibrinogen and 2 U/ml thrombin).

FIG. 4 shows fibroblast cell proliferation (as measured by counts per minute (CPM)) in two different formulations of fibrin over time (formulation A: 50 mg/ml fibrin: 250 U/ml thrombin, and formulation E: 17.3 mg/ml fibrin: 167 U/ml thrombin).

FIG. 5 shows relative reperfusion (as measured by Laser Doppler Imaging (LDI)) in animals treated with a fibrin matrix with and without adipose-derived stem cells at days 1 and 20 following femoral artery ligation. Adipose-derived stem cells have a high degree of CD34 positivity.

FIG. 6 shows results of SDS-PAGE examining crosslinking in fibrin clots at a fibrinogen:thrombin concentration ratio of 17.5:2.

### DETAILED DESCRIPTION OF THE INVENTION

This invention provides a biodegradable, biocompatible fibrin matrix which is used to deliver stem cells. In certain aspects, the invention provides various formulations of fibrin matrix in which to deliver stem cells and increase their retention time at the site of delivery. The fibrin provides a three-dimensional matrix to deliver cells and mimic an in vivo environment in tissues or organs. In certain aspects, the stem cells are CD34+ cells. In various aspects, certain formulations of fibrin are provided by varying fibrinogen to thrombin ratios which alters fibrin-cell response properties. In one embodiment, a formulation of fibrin is provided that sets quickly (in about 90 seconds), but still provides ample time for a technician, scientist, or clinician to prepare the cell/matrix combination, mix thoroughly, and deliver the fibrin matrix formulation into a subject in need thereof. In another embodiment, the fibrin matrix (and dilution buffer) comprises cells. In still another embodiment, the fibrin matrix has a structure that allows for cellular retention and response to the fibrin. In general, the fibrin forms fibers of desired thickness through lateral association, based in part on the number and type of branch points between fibers, to result in a desired porosity. In one aspect, the porosity is such that cells lodge in the fibrin matrix, but not so porous that cells easily come out of the fibrin matrix. A defined porosity and mass per unit length of the fibrin provides the desired retention and 3-D structure that result in cellular response to the fibrin scaffold. The present invention provides such formulations of fibrin and methods for their use.

### Definitions

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that, as used in this specification and the appended claims, the singular forms “a,” “an,” and “the” include plural reference unless the context clearly dictates otherwise.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The phrase “inserting stem cells into a fibrin matrix” is used interchangeably with the phrase “inserting stem cells into a fibrin scaffold.” Both phrases are used interchangeably herein to mean that the cells are inserted or attached to the matrix or scaffold by being coated, encapsulated, embedded, or non-covalently or covalently attached to the matrix or scaffold.

The phrase “a subject in need thereof” is used interchangeably with the term “a subject.” The invention provides methods for the delivery of compositions comprising fibrin scaffold and stem cells in a subject including, but not limited to, an animal subject. In one aspect, the animal subject is a mammal. In a particular aspect, the mammal is a human.

The phrase “delivering the composition comprising fibrin matrix and stem cells” is used herein to encompass any method for administering, injecting, implanting, spraying, and the like, the composition to a subject. As discussed herein below, there are many methods and modes for delivering the fibrin matrix to the subject.

As used herein, the following terms have the meanings ascribed to them unless specified otherwise.

### Fibrin

Fibrin, also known as factor la, is a fibrous, non-globular protein involved in the clotting of blood. More specifically, fibrin is produced from cleavage of fibrinogen, a soluble plasma glycoprotein that is synthesized by the liver and found in blood plasma. Processes in the coagulation cascade activate the zymogen prothrombin to the serine protease thrombin, which is responsible for converting fibrinogen into fibrin. Fibrin molecules then combine to form long fibrin threads that entangle platelets, building up a spongy mass that gradually forms a complex polymer which contracts to form the blood clot. This hardening process is stabilized by a substance known as fibrin-stabilizing factor, or factor XIII.

A fibrin matrix is a network of protein that holds together and supports a variety of living tissues, especially in response to injury. A fibrin matrix exploits the final stage of the coagulation cascade in which fibrinogen molecules are cleaved by thrombin, convert into fibrin monomers and assemble into fibrils, eventually forming fibers in a three-dimensional network. Simultaneously, factor XIII (FXIII) present in the solution is activated by thrombin in the presence of calcium ions to factor XIIIa. The aggregated fibrin monomers and any remaining fibronectin possibly present are cross-linked to form a high polymer by new peptide bonds forming. By this cross-linking reaction, the strength of the clot formed is substantially increased. Generally, the clot adheres well to wound and tissue surfaces, which leads to the adhesive and haemostatic effect. (See U.S. Pat. No. 7,241,603). Therefore, fibrin adhesives are frequently used as two-component adhesives which comprise a fibrinogen complex component together with a thrombin component which additionally contains calcium ions. The terms “fibrin matrix”, “fibrin scaffold”, “fibrin-based scaffold”, “fibrin sealant”, “fibrin glue”, “fibrin gel”, “fibrin adhesive”, and “fibrin clot” are often used interchangeably herein and in the art to refer to a three-dimensional network comprising at least a fibrinogen component and a thrombin component, which can act as a scaffold for cell growth over time.

Such fibrin matrix or fibrin clot is provided naturally by the body after injury, but also can be engineered as a tissue substitute as described herein to speed healing. The fibrin matrix consists of naturally occurring biomaterials composed of cross-linked fibrin network and has a broad use in biomedical applications. For example, it is used to control surgical bleeding, speed wound healing, seal off hollow body organs or cover holes made by standard sutures, and provide slow-release delivery of medications like antibiotics to tissues exposed. Such a fibrin matrix is useful in repairing injuries to the body, and is useful in sites of ischemia. In biomedical research, fibrin matrices have been used to fill bone cavities, and repair neurons, heart valves and the surface of the eye. Fibrin matrices have also been used in the urinary tract, liver, lung, spleen, kidney, and hear. In the present invention, fibrin matrices are used in repairing injury or treating ischemia in any site of the body.

Fibrin sealants are a type of surgical tissue adhesive derived from human and animal blood products. The ingredients in fibrin sealants interact during application to form a stable clot composed of fibrin. Fibrin sealants are used to control surgical bleeding, speed wound healing, seal off hollow body organs or cover holes made by standard sutures, and provide slow-release delivery of medications like antibiotics to exposed tissues. As of about 2003, all fibrin sealants used in the United States are made from blood plasma taken from carefully screened donors and rigorously tested to eliminate hepatitis viruses, HIV-1, and parvovirus. All fibrin sealants in use as of 2003 have two major ingredients, purified fibrinogen protein and purified thrombin enzyme derived from human or bovine (cattle) blood. Many sealants have two additional ingredients, human blood factor XIII and aprotinin, which is derived from cows' lungs. Factor XIII strengthens blood clots by forming cross-links between strands of fibrin. Aprotinin inhibits the enzymes that break down blood clots. Examples of fibrin sealants are described in U.S. Pat. Nos. 5,716,645; 5,962,405; and 6,579,537 and are available in lyophilized, frozen, or non-frozen liquid form. Fibrin sealants have also been designed which lack the aprotinin ingredient (EVICEL, Ethicon, Inc., New Jersey). The invention includes the use of all types of fibrin sealants.

A particular advantage of a fibrin sealant is that the adhesive/gel does not remain at its site of application as a foreign body, but is completely resorbed just as in natural wound healing, and is replaced by newly formed tissue. Various cells, e.g., macrophages and, subsequently, fibroblasts migrate into the gel, lyse, and resorb the gel material and form new tissue. Fibrin sealants have been used to form fibrin gels in situ, and these fibrin gels have been used for delivery of cells and growth factors (Cox et al., Tissue Eng. 10:942-954, 2004; and Wong et al., Thromb. Haemost. 89:573-582, 2003).

In some aspects of the invention, fibrin sealants such as Tisseel® Vapor Heat Solvent Detergent (VHSD) (Baxter International Inc.), a next generation fibrin sealant, are used. Tisseel® VHSD was developed with an added virus inactivation step (solvent/detergent [S/D] treatment) to provide added safety and convenience to the currently licensed Tisseel® product. Tisseel® VHSD is indicated for use as an adjunct to hemostasis in surgeries involving cardiopulmonary bypass and treatment of splenic injuries. In other aspects, fibrin sealants such as Floseal® (Baxter International Inc.) are used. Floseal® is an effective hemostatatic matrix that stops bleeding in 2 minutes or less (median time to hemostasis).

Fibrin sealants are, in one aspect, prepared from separate solutions of thrombin and fibrinogen. The thrombin and fibrinogen solutions are loaded into a double-barreled syringe that allows them to mix and combine. As the thrombin and fibrinogen solutions combine, a clot develops in the same way that it would form during normal blood clotting through a series of chemical reactions known as the coagulation cascade. At the end of the cascade, thrombin breaks up fibrinogen molecules into fibrin molecules that arrange into strands that are then cross-linked by Factor XIII to form a lattice or net-like pattern that stabilizes the clot.

Additional methods for producing fibrinogen-containing preparations that can be used as tissue adhesives include production from cryoprecipitate, optionally with further washing and precipitation steps with ethanol, ammonium sulphate, polyethylene glycol, glycine or beta-alanine, and production from plasma within the scope of the known plasma fractionation methods, respectively (cf., e.g., “Methods of plasma protein fractionation”, 1980, ed.: Curling, Academic Press, pp. 3-15, 33-36 and 57-74, or Blomb ck B. and M., “Purification of human and bovine fibrinogen”, Arkiv. Kemi. 10, 1959, p. 415 f.). Fibrin sealant may also be made using a patient's own blood plasma. For example, the CRYOSEAL (Thermogenesis Corp., Rancho Cordova, Calif.) or VIVOSTAT (Vivolution A/S, Denmark) fibrin sealant systems enable the production of autologous fibrin sealant components from a patient's blood plasma. The components of fibrin sealants are available in lyophilized, deep-frozen liquid, or liquid form.

As discussed above, in various aspects, the fibrin matrix comprises fibrinogen and thrombin. Polymerization time of fibrinogen and thrombin is affected by both the concentration of fibrinogen and thrombin as well as by temperature. Fibrin gel characterization by scanning electron microscopy reveals that thick fibers make up a dense structure at lower fibrinogen concentrations and thinner fibers and a tighter gel can be obtained as fibrinogen concentration increases. In certain aspects, fibrin structure can be modified by the dilution buffered used in preparing the fibrin matrix. Thrombin concentration does not appear to affect polymerization as greatly as fibrinogen, but under defined fibrinogen concentrations, the fiber gel fibers steadily get thinner with increasing concentrations of thrombin. In further aspects, the fibrin matrix may also comprise collagen, fibronectin, and other matrix proteins. In additional aspects, the fibrin matrix is bioabsorbable and biocompatible.

### Cells

The invention includes the use of various types of cells for delivery in the fibrin matrix. In one aspect, stem cells are used. In various aspects, stem cells are autologous, homologous, or heterologous. In a particular aspect, CD34+ cells are used. Cells expressing CD34 (CD34+ cell) are normally found in the umbilical cord and bone marrow as hematopoietic cells, endothelial progenitor cells, endothelial cells of blood vessels, mast cells, a sub-population dendritic cells (which are factor XIIIa negative) in the interstitium and around the adnexa of dermis of skin, as well as cells in soft tissue tumors. The CD34 protein is a member of a family of single-pass transmembrane sialomucin proteins that show expression on early hematopoietic and vascular-associated tissue. Cells observed as CD34+ and CD38- are of an undifferentiated, primitive form; i.e., they are pluripotent hematopoietic stem cells which may be isolated from blood.

In various aspects of the invention, other cell types and cell sources are also used including, but not limited to, differentiated cells (endothelial cells, fibroblasts, and the like), alternate sources of CD34+ cells (adipose-derived stromal cells (ASCs), bone marrow, and cord blood), and other types of stem cells (including mesenchymal stem cells (MSCs) and bone marrow mononuclear cells (BMMNCs)).

In certain embodiments, a cell as used in the invention is selected from the group consisting of totipotent stem cells, pluripotent stem cells, hematopoietic stem cells, adipose stem cells, and any other stem cells with a CD34+ marker. In certain aspects, the cell is isolated via Isolex® technology. In particular aspects, the Isolex® cell is selected from mobilized peripheral blood, bone marrow, or adipose cell source. In more particular aspects, the Isolex® cell of the invention is CD34+. Human CD34+ stem cells are described in U.S. Pat. Nos. 4,965,204; 5,035,994; and 5,130,144. Antibody selection technology (Isolex® 300i, Baxter Healthcare Corp., Deerfield, Ill.), used to isolate, purify, and harvest human CD34+ stem cells from a patient's blood or bone marrow, is described in U.S. Pat. Nos. 5,536,475; 5,968,753; 6,017,719; and 6,251,295. Any means of selecting CD34+ cells can be used in the invention. In certain aspects, Isolex® selection technology is used to isolate CD34+ cells. The invention however is not limited to cells isolated via Isolex® technology as any means of isolating stem cells is included in the invention.

In one aspect, cells are mixed with a pharmaceutically acceptable carrier or diluent in which the cells of the invention remain viable. Pharmaceutically acceptable carriers and diluents contemplated include, without limitation, saline, aqueous buffer solutions, solvents and/or dispersion media. The use of such carriers and diluents is well known in the art. The solution is in one aspect sterile and fluid, and, in some aspects, isotonic. In certain aspects, the solution is stable under the conditions of manufacture and storage and preserved against the contaminating action of microorganisms such as bacteria and fungi through the use of, for example and without limitation, parabens, chlorobutanol, phenol, ascorbic acid, thimerosal, and the like.

### Compositions and Methods

Aspects of the invention provide compositions and methods for regenerative medicine. In one aspect, the invention provides for compositions comprising biocompatible scaffold materials and stem cells.

The components of the fibrin gel are added at appropriate concentrations to provide the type of controlled release desired. Fibrinogen is added in varying concentrations including, but not limited to, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 60 mg/ml, about 70 mg/ml, about 80 mg/ml, about 90 mg/ml, about 100 mg/ml, about 150 mg/ml, and up to about 200 mg/ml (final concentrations in the gels), or in intermediate concentrations as necessary. In certain aspects, the fibrinogen is added at concentrations of about 17.5 mg/ml, about 35 mg/ml, and about 50 mg/ml.

Further, the fibrinogen may be combined with any appropriate concentration of thrombin. Thrombin is added in varying concentrations including, but not limited to, about 1 IU/ml, about 2 IU/ml, about 3 IU/ml, about 4 IU/ml, about 5 IU/ml, about 6 IU/ml, about 7 IU/ml, about 8 IU/ml, about 9 IU/ml, about 10 IU/ml, about 11 IU/ml, about 12 IU/ml, about 13 IU/ml, about 14 IU/ml, about 15 IU/ml, about 16 IU/ml, about 17 IU/ml, about 18 IU/ml, about 19 IU/ml, about 20 IU/ml, about 21 IU/ml, about 22 IU/ml, about 23 IU/ml, about 24 IU/ml, about 25 IU/ml, about 30 IU/ml, about 35 IU/ml, about 40 IU/ml, about 45 IU/ml, about 50 IU/ml, about 60 IU/ml, about 70 IU/ml, about 80 IU/ml, about 90 IU/ml, about 100 IU/ml, about 110 IU/ml, about 120 IU/ml, about 130 IU/ml, about 140 IU/ml, about 150 IU/ml, about 160 IU/ml, about 170 IU/ml, about 180 IU/ml, about 190 IU/ml, about 200 IU/ml, about 225 IU/ml, about 250 IU/ml, about 275 IU/ml, about 300 IU/ml, or in intermediate concentrations as necessary. In certain aspects, thrombin is added at concentrations of about 2 IU/ml, 4 IU/ml, 8 IU/ml, 50 IU/ml, 167 IU/ml, and 250 IU/ml.

In some aspects, the compositions of the invention comprise fibrin matrix formulations (thrombin to fibrinogen ratios) in ratios ranging from about 0.001 to about 100.0. In another aspect, thrombin to fibrinogen ratios range from about 0.01 to about 10.0. In various aspects, the ratio is about 0.04, or about 0.05, or about 0.11. The invention includes, but is not limited to, the following fibrinogen to thrombin ratios: about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, about 15, about 20, about 25, about 30, about 35, about 40, about 45, about 50, about 55, about 60, about 65, about 70, about 75, about 80, about 85, about 90, about 95, and about 100, or in intermediate ratios as necessary.

In some aspects of the invention, the fibrin clot comprises fibrinogen at a final concentration from about 1 mg/ml to about 100 mg/ml and thrombin at a final concentration from about 1 IU/ml to about 250 IU/ml. In particular aspects of the invention, fibrin matrix formulations (final concentrations of fibrinogen (mg/ml) to thrombin (international units (IU) or units (U)/ml) are about 17.5 mg/2U, about 12 mg/8U, about 5 mg/2U, about 12 mg/2U, about 5 mg/4U, about 12 mg/4U, about 17.5 mg/4U, about 5 mg/8U, about 17.5 mg/8U, about 9 mg/50U, about 17.5 mg/167U, about 50 mg/2U, about 50 mg/250U and about 17.3 mg/167U.

In various aspects of the invention, synthetic polymers can be mixed with fibrin to form a biodegradable hybrid scaffold. Such synthetic polymers include, but are not limited to, polymers such as poly(lactide) (PLA), poly(glycolic acid) (PGA), poly(lactide-co-glycolide) (PLGA). poly(caprolactone), polycarbonates, polyamides, polyan hydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof.

In one aspect, a composition of the invention comprises fibrin matrix and CD34+ stem cells. In a particular aspect, a composition of the invention comprises fibrin matrix and Isolex®-derived CD34+ stem cells (Isolex® cells). In certain aspects, Isolex® cells are from mobilized peripheral blood, bone marrow, adipose tissue, or any CD34+ cell isolated on the Isolex® system.

In further embodiments, the Isolex® cell or other stem cell may be combined with another stem cell being selected from the group consisting of totipotent stem cells, pluripotent stem cells, hematopoietic stem cells, adipose stem cells, and any other stem cells. In certain aspects, the Isolex® cell or other stem cell is combined with either non-hematopoietic stem cells such as mesenchymal—early or late precursor—cells and fibrin, collagen, or PEG.

In another aspect, the invention provides for cell delivery by being coated, encapsulated, embedded, and non-covalently and covalently attached to the scaffold materials which become incorporated or attached to the tissue or organ site for the purpose of reversing ischemia and producing cell, tissue, and/or organ regeneration. In one aspect, compositions comprising fibrin matrix and CD34+ cells are administered with CD34+ cells in a concentration ranging from about 1,000 to about 2,000,000 cells per volume (1 mL) of fibrin clot. In another aspect, CD34+ cells are administered in a concentration ranging from about 10,000 to about 1,000,000 cells per 1 mL of fibrin clot. In various aspects, CD34+ cells are administered in a concentration ranging of about 10,000 cells per clot, about 15,000 cells per clot, about 20,000 cells per clot, about 25,000 cells per clot, about 30,000 cells per clot, about 35,000 cells per clot, about 40,000 cells per clot, about 45,000 cells per clot, about 50,000 cells per clot, about 55,000 cells per clot, about 60,000 cells per clot, about 65,000 cells per clot, about 70,000 cells per clot, about 75,000 cells per clot, about 80,000 cells per clot, about 85,000 cells per clot, about 90,000 cells per clot, about 95,000 cells per clot, about 100,000 cells per clot, about 125,000 cells per clot, about 150,000 cells per clot, about 200,000 cells per clot, about 225,000 cells per clot, about 250,000 cells per clot, about 275,000 cells per clot, about 300,000 cells per clot, about 325,000 cells per clot, about 350,000 cells per clot, about 375,000 cells per clot, about 400,000 cells per clot, about 450,000 cells per clot, about 475,000 cells per clot, about 500,000 cells per clot, about 525,000 cells per clot, about 550,000 cells per clot, about 575,000 cells per clot, about 600,000 cells per clot, about 625,000 cells per clot, about 650,000 cells per clot, about 675,000 cells per clot, about 700,000 cells per clot, about 725,000 cells per clot, about 750,000 cells per clot, about 775,000 cells per clot, about 800,000 cells per clot, about 825,000 cells per clot, about 850,000 cells per clot, about 875,000 cells per clot, about 900,000 cells per clot, about 925,000 cells per clot, about 950,000 cells per clot, about 975,000 cells per clot, about 1,000,000 cells per clot, about 1,500,000 cells per clot, about 2,000,000 cells per clot, about 3,000,000 cells per clot, about 4,000,000 cells per clot, about 5,000,000 cells per clot, about 6,000,000 cells per clot, about 7,000,000 cells per clot, about 8,000,000 cells per clot, about 9,000,000 cells per clot, about 10,000,000 cells per clot, about 15,000,000 cells per clot, about 20,000,000 cells per clot, about 25,000,000 cells per clot, about 30,000,000 cells per clot, about 35,000,000 cells per clot, about 40,000,000 cells per clot, about 50,000,000 cells per clot, and up to about 100,000,000 cells per clot. One skilled in the art will appreciate that the appropriate levels of cells for treatment will thus vary depending, in part, upon the volume of the scaffold, the tissue cite to which scaffold is delivered, the indication for which the scaffold is being used, the route of administration, and the size (body weight, body surface or organ size) and condition (the age and general health) of the patient. Accordingly, the clinician may titer the dosage and modify the number of cells delivered to obtain the optimal therapeutic effect.

### Delivery

In aspects of the invention, the scaffold comprising stem cells is delivered to a patient by several means. In some aspects, the scaffold comprising stem cells is delivered intramuscularly, intraperitoneally, intracranially, between tissue components such as fractured or broken bone or cartilage. In other aspects, the fibrin scaffold comprising stem cells is delivered parenterally through injection by intravenous, intracerebral (intraparenchymal), intracerebroventricular, intracerebrospinal, intraocular, intraarterial, intraarticular, intraportal, intrarectal, intranasal, or intralesional routes. In addition, a fibrin scaffold of the invention can be introduced for treatment into a mammal by other modes, such as but not limited to, intratumor, topical, subconjunctival, intrabladder, intravaginal, epidural, intracostal, intradermal, inhalation, transdermal, transserosal, intrabuccal, dissolution in the mouth or other body cavities, instillation to the airway, insuflation through the airway, injection into vessels, tumors, organ and the like, and injection or deposition into cavities in the body of a mammal.

In another aspect, delivery of the stem cells in the fibrin matrix can be targeted to any site in the body. In certain aspects, the target body site is in the nerves, liver, kidney, heart, lung, eye, organs of the gastrointestinal tract, skin, and/or brain. In more particular aspects, the target body site is the heart, eye, brain, and/or kidney.

The invention includes many various vehicles for delivering stem cells in the fibrin matrix into a subject. In one aspect, direct injection by needle and syringe is used. In certain aspects, direct injection includes mixing fibrin and cells in the syringe immediately prior to injection in a subject. In other aspects, the invention includes the use of a cell mixing chamber (between syringe and needle) to increase mixing. In various aspects, the invention includes delivery of the stem cells in the fibrin matrix via an injection catheter (for deeper tissue delivery), a spray for surface delivery, or by implanting pre-made fibrin (subcutaneous or deeper within tissue beds). In certain instances, the implanting can be carried out via injection or via surgery.

Where desired, the fibrin scaffold is administered by bolus injection or continuously by infusion, or by implantation device. Alternatively or additionally, the fibrin scaffold is administered locally via implantation of a membrane, sponge, or another appropriate material on to which the scaffold has been absorbed or encapsulated. Where an implantation device is used, the device may be implanted into any suitable tissue or organ, and delivery of the scaffold may be via diffusion, timed release bolus, or continuous administration.

In certain aspects, it may be desirable to use or administer the fibrin scaffold in an ex vivo manner. In such instances, cells, tissues, or organs that have been removed from the patient are exposed to compositions after which the cells, tissues and/or organs are subsequently implanted back into the patient.

In other aspects of the invention, additional ways of delivering fibrin scaffold to a subject will be evident to those skilled in the art, including formulations involving scaffold in sustained or controlled delivery formulations. Techniques for formulating a variety of other sustained or controlled delivery means are known to those skilled in the art.

In one aspect, delivery in a subject is made by injection with a syringe and needle. In a certain aspect, delivery is made with a syringe and 25-gauge needle. However, various sizes of syringes and needles are also used for delivery. In various aspects, the syringe may range in size between 0.5 to 100 cc. However, the size of the syringe is not limiting with respect to the invention. Other sizes can also be used. In further aspects, the size of the needle may range between a 16 and 30 gauge needle. Like the syringe, needle size is not limiting with respect to the invention.

A single bolus injection may be given by intravenous infusion or by direct injection, using a syringe. This mode of administration may be desirable in surgical patients, if appropriate, such as patients having cardiac surgery, e.g., coronary artery bypass graft surgery and/or valve replacement surgery. In these patients, a single bolus infusion of scaffold can be administered. (Note that the amount of drug administered is based on the weight and condition of the patient and is determined by the skilled practitioner.) Shorter or longer time periods of administration can be used, as determined to be appropriate by one of skill in this art.

In cases in which longer-term delivery of a scaffold comprising stem cells is desirable, intermittent administration can be carried out. In these methods, a loading dose is administered, followed by either (i) a second loading dose and a maintenance dose (or doses), or (ii) a maintenance dose or doses, without a second loading dose, as determined to be appropriate by one of skill in this art.

To achieve further delivery of the scaffold composition in a patient, a maintenance dose (or doses) of the fibrin scaffold can be administered. Maintenance doses can be administered at levels that are less than the loading dose(s), for example, at a level that is about ⅙ of the loading dose. Specific amounts to be administered in maintenance doses can be determined by a medical professional, with the goal that the scaffold comprising stem cells is at least maintained at the target cite for a period of time. Of course, maintenance doses can be stopped at any point during this time frame, as determined to be appropriate by a medical professional.

In other aspects of the invention, delivery is made with a catheter. Delivery by catheter can be carried out by using products (for example, infusion pumps and tubing) that are widely available in the art. One criterion that is important to consider in selecting a catheter and/or tubing to use in these methods is the impact of the material of these products (or coatings on these products) on the scaffold comprising stem cells. Additional catheter-related products that can be used in the methods of the invention can be identified by determining whether the material of the products alters the scaffold, under conditions consistent with those that are used in drug administration.

### Dosing

The invention includes the use of various dosing parameters. In one aspect, cells are dosed per kilogram body weight of a subject in need thereof. For example, in critical limb ischemia (CLI), multiple injections are made surrounding and/or upstream of the ischemic region. In various aspects, a subject receives multiple doses or multiple instances of treatment. In certain aspects, it is possible to re-dose a subject for increased or prolonged effects (weeks, months, or even years into the future). In dosing, the fibrin displaces a set amount of volume in the muscle and/or surrounding areas. Therefore, it is important to monitor the volume of cell/matrix that can be injected. In one aspect, the maximum amount of cells to be delivered per fibrin sample is dictated by the volume (size) of the fibrin components used as well as the area of treatment and the size of the subject. In some aspects, larger subjects tolerate larger volumes of fibrin, making increased dosing or volume of cells desirable.

An “effective amount” or an “amount effective” refers to the amount of fibrin matrix and/or amount of stem cells to achieve an observable change in a subject. An “effective amount” or an “amount effective” of a composition to be employed therapeutically will depend, for example, upon the therapeutic context and objectives. One skilled in the art will appreciate that the appropriate dosage levels for treatment will thus vary depending, in part, upon the tissue site to which scaffold is delivered, the indication for which the scaffold is being used, the route of administration, and the size (body weight, body surface or organ size) and condition (the age and general health) of the patient. Accordingly, the clinician may titer the dosage and modify the route of administration to obtain the optimal therapeutic effect.

An exemplary regimen includes, for example and without limitation, administration of from about 1 to about 2,000,000 cells per fibrin clot given in daily doses or in equivalent doses at longer or shorter intervals, for example, every other day, twice weekly, weekly, monthly, semi-annually, or even twice or three times daily. In certain aspects, multiple clots are delivered. In some aspects of the invention, the dose of fibrin scaffold comprising cells is delivered in multiple doses. In one aspect, doses are delivered in about 1 to 50 subdelivery components. In certain aspects, the range of total doses is from about 1 to about 20. In other aspects, the range of doses is from about 1 to about 10. In various aspects, the range of doses is from about 1 to about 5. In more particular aspects, the range of doses is from about 1 to about 3.

The frequency of dosing will depend upon multiple parameters. Typically, a clinician will administer the composition until a dosage is reached that achieves the desired effect. The scaffold composition may therefore be administered as a single dose, or as two or more doses (which may or may not contain the same amount of stem cells) over time. In further aspects, the fibrin scaffold is administered via a continuous infusion via implantation device or catheter. Further refinement of the appropriate dosage is routinely made by those of ordinary skill in the art and is within the ambit of tasks routinely performed by a clinician or a person of ordinary skill in the art. Appropriate dosages may be ascertained through use of appropriate dose response data.

### Methods of Treatments and Uses

In aspects of the invention, the fibrin matrix comprising stem cells is used in treating ischemia or for tissue regeneration after tissue damage or loss resulting from disease or injury. For example, tissue damage due to ischemia due to blood flow loss, lacerations, extremes of temperature, trauma, or metabolic or genetic disease, is one of many various conditions or diseases which can benefit from treatment with stem cells in a fibrin scaffold. Other diseases include cardiovascular disease, diabetes, autoimmune diseases, stroke, brain and/or spinal cord injury, burn injury, bone defects, renal ischemia, and macular degeneration. In another aspect, the scaffold comprising cells is used to treat an ischemic or a cirrhotic liver. In various aspects, the invention includes uses of a composition comprising fibrin matrix and stem cells for the manufacture of a medicament for treating a localized site of injury or disease, for enhancing vascularization to a localized site, or for treating ischemia.

In certain aspects, the scaffold of the invention is used to treat critical limb ischemia (CLI). CLI represents a syndrome that is associated with a particularly adverse natural history. Although clinicians increasingly recognize that peripheral arterial disease (PAD) includes a broad range of clinical syndromes, CLI is associated with very adverse short-term limb and systemic cardiovascular outcomes. CLI is not a specific disease per se, but rather represents a syndrome that may develop from many fundamentally distinct pathophysiological processes, including advanced atherosclerosis, thromboembolism or atheroembolism, in situ thrombosis, and the arteritides, such as thromboangiitis obliterans (also known as TAO or Buerger disease).

### Kits

In a further aspect, the invention includes a kit for preparing the fibrin scaffold comprising stem cells and administering it to a subject in need thereof. The fibrin scaffold may be advantageously provided in kit form including separately packaged amounts of fibrin sealant and thrombin. In another aspect, the kit may further comprise stem cells from another source or an agent for isolating the subject's own stem cells. Alternatively, a kit can include an additional biological agent that can be delivered in the fibrin matrix or in conjunction with the administration of the fibrin matrix. In an exemplary embodiment of a kit, each component of the kit is packaged separately in sterile packaging or in packaging susceptible to sterilization. The biological agents, including the fibrin component, the thrombin component, or the cells, may be provided in a container such as a glass or plastic vial and may further be carried or suspended in a liquid storage medium suitable for maintaining cells or other biological compounds. The kit may optionally further include one or more syringes, catheters or other delivery device(s) for introducing the fibrin scaffold into the subject. Kits may optionally further include one or more additional containers each storing a pharmaceutical agent that may be added to the fibrin scaffold. The kit further includes, for example, printed instructions for making and using the fibrin scaffold. All elements of the kit are provided together in suitable amounts in a box or other suitable packaging.

### EXAMPLES

The invention is described in more detail with reference to the following non-limiting examples, which are offered to more fully illustrate the invention, but are not to be construed as limiting the scope thereof. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention. All patents and publications mentioned herein are incorporated by reference.

Example 1 describes a method for isolating mononuclear cells from umbilical cord blood. Example 2 provides a method of selecting and purifying CD34+ cells from umbilical cord blood mononuclear cells (MNC). Example 3 describes how to prepare fibrin gel from Tisseel® VHSD. Example 4 describes how to prepare fibrin gel from Floseal®. Example 5 describes experiments carried out to determine parameters to optimize scaffold composition by measuring polymerization rates and cell viability over time. Example 6 describes further experiments examining CD34+ cell proliferation in a fibrin matrix over time. Example 7 shows CD34+ cell viability in various concentrations of Tisseel® over time. Example 8 shows that CD34+ cells in a fibrin scaffold enhanced revascularization of ischemic hind limbs in preclinical studies of critical limb ischemia. Example 9 shows that crosslinking occurs in a fibrin clot at a fibrinogen:thrombin ratio of 17.5:2.

### Example 1

### Preparation of Mononuclear Cells for CD34+ Cell Selection

This Example describes how to reduce red cells from whole blood (approximately 90%) and how to isolate mononuclear cells (MNC) in umbilical cord blood. Single or pooled cord blood (CB) samples ranging from 48-72 hours old and 40-100 mls in volume were obtained with parental consent. MNC from CB were prepared at a dilution of 1 in 5 with 1 ml of 6% hetastarch (Baxter Healthcare Corp., Deerfield, Ill.) to every 5 ml of unseparated CB. The mixture was allowed to settle for a minimum of 1 hour at room temperature. The plasma fraction containing MNC was then transferred into a 50 ml conical tube and pelleted by centrifugation for 7 to 10 minutes. MNC were resuspended with approximately 35 mls of calcium/magnesium-free phosphate buffered saline (CMF-PBS) (Lonza Corp., Walkersville, Md.) and then underlayed with 12-15 mls of Histopaque-1077 (Sigma Chemical, St. Louis, Mo.). After 20-30 minutes of centrifugation at 400×g, the MNC interface layer was isolated and washed once in 20-30 mls of CMF-DPBS. CD34+ cells were then selected from the MNC as described in Example 2 below.

### Example 2

### Selection of CD34+ Cells from Umbilical Cord Blood

This Example describes how to select and purify CD34+ cells from umbilical cord blood MNC using the EasySep® human CD34+ cell selection kit (Stem Cell Technologies, Vancouver, Canada). The CD34 antibody was added at 100 μl/ml per every 1×10⁸ cells or 2×10⁸ cells. The antibody and cells were mixed well, and then incubated at room temperature for 15 minutes. Magnetic nanoparticles were then added at 50 μl/ml. The cells and nanoparticles were mixed well, and then incubated at room temperature for 10 minutes. The cell/nanoparticle mixture was resuspended with Buffer (+EasySep Kit) at 2.5 ml. The cells were placed in a tube and then into the EasySep® magnet and allowed to set for 5 minutes. In one continuous motion, the magnet and tube were inverted while pouring off the supernatant fraction. The magnetically labeled CD34+ cells remained inside the tube, held by the magnetic field of the magnet while the unwanted (non-magnetic cells) were washed away with the supernatant fraction. This wash and magnetic bead capture steps were repeated for a total of five times. After the washes, the tube was removed from the magnet and the cells were resuspended in an appropriate amount (approximately 1 ml) of X-VIVO 10® cell culture medium (Lonza Corp., Walkersville, Md.). CD34+ cells were then counted on a hemacytometer.

### Example 3

### Preparation of Fibrin Gel from Tisseel® VHSD

This Example describes how to prepare fibrin gel from Tisseel® VHSD (Baxter International Inc.), a next generation fibrin sealant, developed with an added virus inactivation step (solvent/detergent [S/D] treatment) to provide added safety and convenience to the currently licensed Tisseel® product. The fibrinogen component Tisseel® VHSD (sealer protein) was resuspended with 5 mls of aprotinin and then placed in the fibrinotherm at 37° C. until dissolved. Undiluted stock was prepared at a concentration of 100 mg/ml of fibrinogen and diluted 1:4 to a concentration of 25 mg/ml with fibrinogen dilution buffer (FDB). FDB contains 3000 KIU/ml aprotinin, 25 mM sodium citrate, 48 mM sodium chloride, 333 mM glycine, and 15 g/L human serum albumin. Diluted fibrinogen was then dispensed at 100 μl into individual wells of a 24-well plate. Stock thrombin was prepared in 5 mls of calcium chloride to 500 U/ml and then diluted to 4 U/ml in thrombin dilution buffer (TDB) (40 μl of stock in 5 mls buffer). Each well containing 100 μl of fibrinogen received 100 μl of diluted thrombin to give a final thrombin concentration of 2 U/ml and fibrinogen at 12.5 mg/ml per well. The plated fibrin gel was then incubated at room temperature for 1 hour and then rinsed with 1 ml of Dulbecco's PBS.

### Example 4

### Preparation of Floseal®

This Example describes how Floseal® (Baxter International Inc.), an effective hemostatatic matrix that stops bleeding in 2 minutes or less (median time to hemostasis), was prepared for use in various aspects of the invention. Floseal® was prepared by mixing 5 mls of TDB in a 5 ml syringe. An additional syringe containing the Floseal® gelatin particles was attached via a luer-loc connector to the 5 ml syringe containing TDB, and the contents of both syringes were “swished” back and forth 20 times to mix well. Floseal®/TDB was allowed to sit for 10 minutes at room temperature or until ready for use in further assays.

### Example 5

### Determining Parameters to Optimize Scaffold Composition by Measuring Polymerization Rates and Cell Viability Over Time

Initial polymerization rates were determined by measuring the optical density of the fibrin over time. Fiber thickness, lateral formations, and porosity were examined using a scanning electron microscope (SEM).

Cell viability (i.e. cell quantification) was determined using flow cytometry with a dye (7-Amino-Actinomycin D (7-AAD) Viability Dye, Beckman Coulter) that only permeates the cell when its membrane is compromised, thus allowing discrimination of viable from non viable cells using flow cytometry. Other methods of measuring cell viability over time in a fibrin scaffold are described in Bensaid et al. (Biomaterials 24:2497-2502, 2003).

Various formulations of fibrin (final concentrations of fibrinogen (mg/ml) to thrombin (international units (IU) or units (U), as used herein)) were tested as follows: 17.5 mg/2U, 12 mg/8U, 5 mg/2U, 12 mg/2U, 5 mg/4U, 12 mg/4U, 17.5 mg/4U, 5 mg/8U, 12 mg/8U, 17.5 mg/8U, 9 mg/50U, 17.5 mg/167U, and 50 mg/2U.

Diluents used for polymerization studies are set out below. Fibrinogen dilution buffer (FDB) can be used optionally with or without niacinamide.

### Fibrinogen Dilution Buffer (FDB) for Tisseel® VHSD

### 3000 U/mL Aprotinin

### 25 mM Na₃ Citrate

### 50 mM Niacinamide

### 100 mM Histidine

### 15 g/L HSA

pH 7.3

### Thrombin Dilution Buffer (TDB) for Tisseel® VHSD

### 40 mM CaCl₂×2 H₂O

### 64 mM NaCl

### 50 g/L HSA

pH 7.3

### Baxter Phosphate-Buffered Saline

(PBS) (code EDR 9865)

### 137 mM NaCl

### 2.68 mM KCl

### 3.21 mM Na₂HPO_{4×12} H20

pH 7.2

### Tris-Buffered Saline (TBS)

### 20 mM Tris

### 500 mM NaCl

pH 7.4

### 30 mM CaCl₂ in TBS

pH 6.5

### X-VIVO 10 Media BioWhittaker (Lonza)

This is a commercially available media often used to culture hematopoietic stem cells. Supplemented with 20 ng/mL each of thrombopoietin (TPO), stem cell factor (SCF), and Fms-like tyrosine kinase (Flt3L). Use of this media is known for the in vitro culture of stem cells.

Table 1 shows the polymerization rates (time ranges) of three concentrations of fibrin (17.5/2; 12/8; and 5/2) in four different diluents (FDB/TDB; X-VIVO/X-VIVO; PBS/PBS; and TBS/30 mM CaCl₂) in TBS. Fibrinogen is expressed in mg/ml and thrombin is expressed in U/ml.

TABLE I

| | Fibrin Concentration | | | |
|---|---|---|---|---|
| | Ratio of Fibrinogen (mg/ml)/Thrombin (units) | | | |
| | | 17.5/2 U | 12/8 U | 5/2 U |
| Diluent | FDB/TDB | 48-78 sec | 78-144 sec | 81-126 sec |
| | X-VIVO/ | 90-129 sec | 81-108 sec | 78-102 sec |
| | X-VIVO | | | |
| | PBS/PBS | 99-147 sec | 78-114 sec | 81-123 sec |
| | TBS/30 mM | 75-165 sec | 42-111 sec | 130->180 |
| | CaCl₂ in TBS | | | |

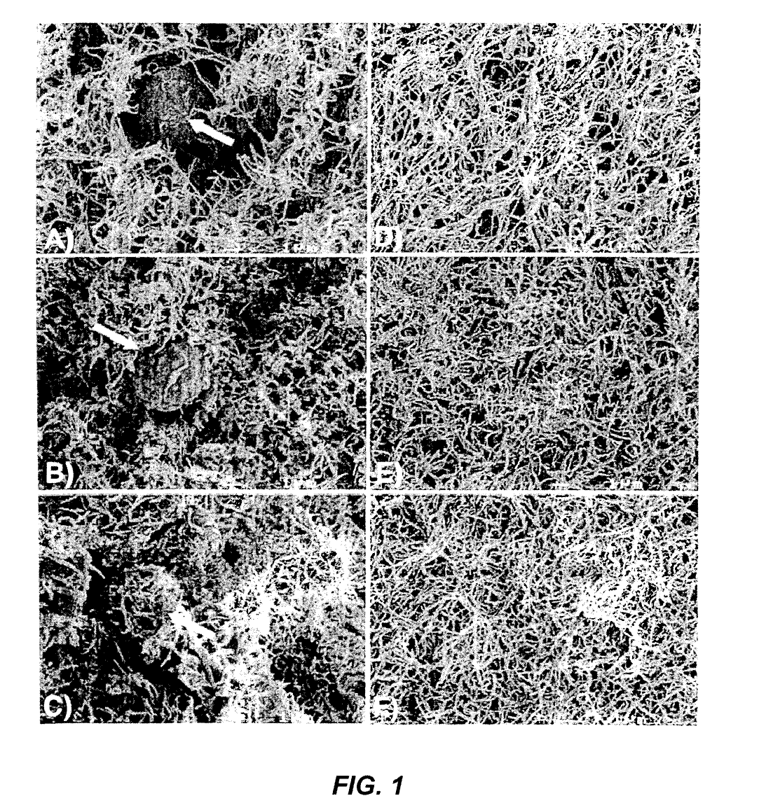

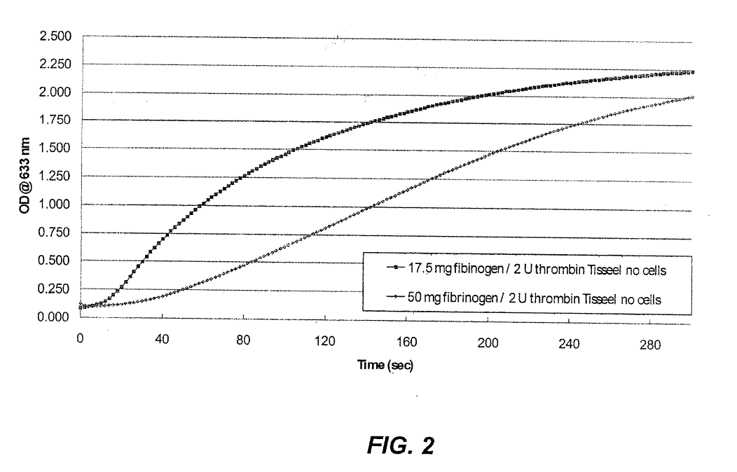

FIG. 1 shows scanning electron micrographs (SEM) of a CD34+ stem cell (see arrows) in various concentrations of fibrin matrix. FIGS. 1 (A-C) show SEMs of cells in fibrin matrix with fibrinogen and thrombin diluted in PBS: (A) 17.5 mg/ml fibrinogen and 2 U/ml thrombin; (B) 35 mg/ml fibrinogen and 2 U/ml thrombin; and (C) 50 mg/ml fibrinogen and 2 U/ml thrombin, all diluted using PBS. FIGS. 1 (D-F) show SEMs of fibrin matrix without cells with fibrinogen and thrombin diluted in PBS: (D) 17.5 mg/ml fibrinogen and 2 U/ml thrombin; (E) 35 mg/ml fibrinogen and 2 U/ml thrombin; and (F) 50 mg/ml fibrinogen and 2 U/ml thrombin, all diluted using PBS. FIG. 2 shows polymerization curves generated for fibrin clots formed with (1) 17.5 mg/ml fibrinogen and 2 U/ml thrombin and (2) 50 mg/ml fibrinogen and 2 U/ml thrombin. Tables 2A, B, and C shows cell viability in 17.5/2 fibrin at various cell concentrations per clot and in various diluents over time.

TABLE 2A

| 17.5/2 | | | | | | | |
|---|---|---|---|---|---|---|---|
| formulation | | | | | | | |
| Expt | Diluent | Mixing mechanism | Cells per clot | Day 0 | Day 1 | Day 4 | Day 7 |
| 13-Mar-08 | FDB/TDB | mixer | 25,000 | NA | 44% | 0% | N/A |
| 13-Mar-08 | FDB/TDB | mixer | 50,000 | NA | 63% | 0% | N/A |
| 27-Mar-08 | FDB/TDB | mixer | 50,000 | 78% | N/A | N/A | 0% |
| 27-Mar-08 | TBS/CaCl2 | mixer | 50,000 | 46% | N/A | N/A | 0% |
| 10-Apr-08 | FDB/TDB | mixer | 50,000 | 96% | 87% | N/A | 0% |
| 10-Apr-08 | PBS | mixer | 50,000 | 98% | 82% | 84% | 86% |
| 17-Apr-08 | FDB/TDB | mixer | 50,000 | 96% | 80% | 0% | 0% |
| 17-Apr-08 | FDB2/TDB2 | mixer | 50,000 | 86% | 80% | 0% | 0% |
| 17-Apr-08 | PBS | mixer | 50,000 | 95% | 58% | 0% | 0% |
| 1-May-08 | PBS | mixer | 50,000 | 96% | 65% | 83% | 72% |
| 8-May-08 | PBS | mixer | 50,000 | 98% | 87% | 79% | N/A |
| 8-May-08 | PBS | mixer | 300,000 | 97% | 81% | 84% | 76% |
| 8-May-08 | PBS | mixer | 600,000 | 98% | 78% | 71% | 65% |
| 12-Jun-08 | PBS | manual | 600,000 | NA | 93% | 78% | 81% |
| 19-Jun-08 | PBS | manual | 600,000 | NA | 85% | 69% | 53% |

TABLE 2B
Swooshing method with luer connectors and injection through
cannula

| | | | | |
|---|---|---|---|---|
| 1-May-08 | PBS | swooshing | 50,000 | 96% |

Swooshing method with luer connectors and injection through
26G needle

| | | | | |
|---|---|---|---|---|
| 1-May-08 | PBS | swooshing | 50,000 | 95% |

FDB/TDB = fibrinogen dilution buffer/thrombin dilution buffer
FDB2/TDB2 = 2nd batch of fibrinogen dilution buffer/thrombin dilution buffer

TABLE 2C

Days

| | | | | |
|---|---|---|---|---|
| Experiment | 0 | 1 | 4 | 7 |

300,000 cells/0.3 mL clot

| | | | | |
|---|---|---|---|---|
| May 8 Exp | 96.99 | 81.08 | 84.08 | 75.785 |

600,000 cells/0.3 mL clot

| | | | | |
|---|---|---|---|---|
| May 8 Exp | 97.57 | 78.21 | 71.24 | 64.81 |
| June 13 Exp | N/A | 92.80 | 77.70 | 85.35 |
| June 20 Exp | N/A | 84.81 | 68.85 | 52.89 |
| Average | 97.57 | 85.27 | 72.59 | 67.68 |
| Std Dev | | 7.30 | 4.58 | 16.42 |

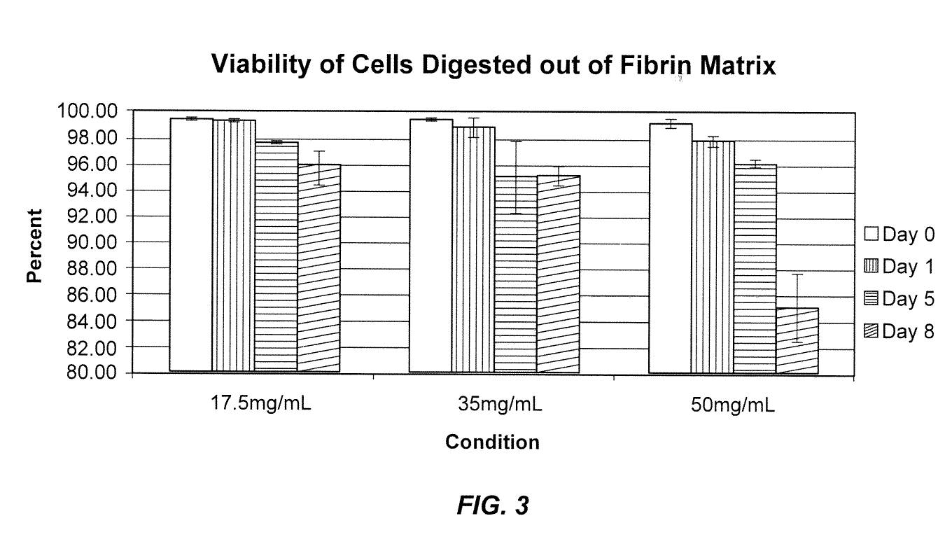

FIG. 3 shows cell viability over 8 days in fibrin formulations of 17.5/2 (17.5 mg/ml fibrinogen and 2 U/ml thrombin), 35/2 (35 mg/ml fibrinogen and 2 U/ml thrombin), and 50/2 (50 mg/ml fibrinogen and 2 U/ml thrombin). After 8 days, cells retained greater viability in the 17.5/2 formulation.

In one aspect of the invention, the initial polymerization rates, pore size, fiber thickness, and cell viability indicate that the fibrin diluted in PBS, with a final concentration of 17.5 mg/mL fibrinogen and 2 U/mL thrombin were very optimal over the 7 day period. However, it is contemplated that other concentrations of fibrin can also be used in various aspects of the invention.

### Example 6

### Cell Proliferation in a Fibrin Matrix

Fibrin gels were prepared as described. The fibrin gels were incubated until they were solid (approximately 1 hour) and then rinsed with 1 ml of Dulbecco's phosphate-buffered saline (DPBS) per well. Diluent was then added at 600 μl per well above each of the fibrin gels. Cells were then added at 100 μl per insert or approximately 100,000 cells per insert above clots according to assay instructions. The plates were incubated overnight at 37° C. and 5% CO₂. Following incubation, non-adherent cells (cells that were not adherent in the fibrin gels) were removed from each of the wells and then counted on a hemacytometer. The cells inside each of the gels were then recovered. Diluted bovine trypsin was prepared at 1 to 4 dilutions in DPBS (0.5 ml trypsin with 2 mls DPBS). The diluted trypsin was then dispensed at 200 μl per well and then incubated at 37° C. until the fibrin gels had dissolved. To each well, 100 μl of FBS was added to stop enzyme activity. The recovered cells were then counted on a hemacytometer.

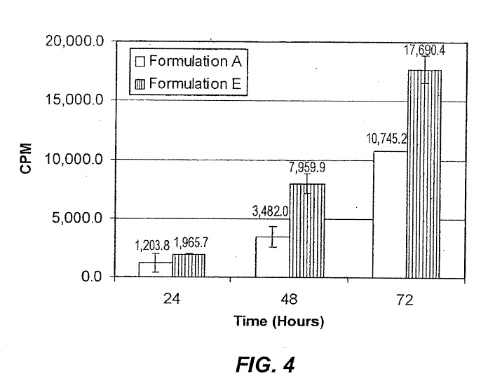

To determine optimal scaffold concentrations, fibroblasts were cultured for 72 hours in fibrin scaffolds prepared by using fibrinogen/thrombin solution volume rations of 50/250 (Formulation A: 50 mg/ml fibrin: 250 U/ml thrombin) and 17.3/167 (Formulation E: 17.3 mg/ml fibrin: 167 U/ml thrombin), respectively. FIG. 4 shows fibroblast cell proliferation (as measured by counts per minute (CPM)) in two different formulations of fibrin (formulation A: 50 mg/ml fibrin: 250 U/ml thrombin, and formulation B: 17.3 mg/ml fibrin: 167 U/ml thrombin). These data showed that fibroblasts proliferated better and over a longer time period when they were cultured at a lower concentration of fibrinogen and thrombin (Formulation E), thereby leading to a decision to modify formulations in further experiments. Cells cultured in a fibrin matrix of Formulation E also showed greater cell proliferation at 24 and 72 hours.

### Example 7

### CD34+Cell Viability in Fibrin Matrix

To examine changes in CD34+ cell viability in fibrin matrix over time, fibrinogen was used at three different concentrations (17.5 mg/mL, 35 mg/mL, and 50 mg/mL) with a constant concentration of thrombin (2 U/mL). On days 0, 1, 5, and 8, fibrin matrices were digested with trypsin-EDTA (0.25%) and an event count was collected via flow cytometry to determine the potential increase or decrease in cellular events. The viability of the cellular events present was determined by an intracellular dye, 7-AAD, as described previously.

The experiments showed that there was a decrease in cellular (gated) events over time at all three fibrin concentrations (see Table 3). Table 3A shows means and 3B shows standard deviations. The 17.5 mg/mL concentration of fibrin demonstrated a slightly increased number of cellular events when compared to fibrin at concentrations of 35 mg/mL and 50 mg/mL at a majority of the time points.

TABLE 3A
Gated Events per 30 second
Acquisition Time

| Gated Events (Means) | Day 0 | Day 1 | Day 5 | Day 8 |
|---|---|---|---|---|
| 17.5 mg/mL | 35784 | 25404 | 6422 | 9099 |
| 35 mg/mL | 26270 | 13201 | 9630 | 7714 |
| 50 mg/mL | 32297 | 21151 | 9112 | 3040 |

TABLE 3B

| Gated Events | | | | |
|---|---|---|---|---|
| (SD) | Day 0 | Day 1 | Day 5 | Day 8 |
| 17.5 mg/mL | 3515 | 2128 | 223 | 4185 |
| 35 mg/mL | 684 | 2988 | 598 | 1672 |
| 50 mg/mL | 217 | 7666 | 445 | 240 |

Cell viability decreased slightly over time at all three fibrin concentrations (see Table 4). Table 4A shows means and 4B shows standard deviations. After eight days, the viability of the 50 mg/mL concentration was 85% while cell viability at the other two concentrations (17.5 mg/mL and 35 mg/mL) was greater than 95%. At all of the other time points, the viability at all three fibrin concentrations was greater than 95%.

TABLE 4A
Cell Viability (%) after Tisseel ®
Digestion

| Cell Viability (Means) | Day 0 | Day 1 | Day 5 | Day 8 |
|---|---|---|---|---|
| 17.5 mg/mL | 99.56 | 99.49 | 97.86 | 95.93 |
| 35 mg/mL | 99.55 | 98.97 | 95.24 | 95.35 |
| 50 mg/mL | 99.20 | 97.90 | 96.24 | 85.11 |

TABLE 4B

| Cell Viability | | | | |
|---|---|---|---|---|
| (SD) | Day 0 | Day 1 | Day 5 | Day 8 |
| 17.5 mg/mL | 0.042 | 0.085 | 0.035 | 1.245 |
| 35 mg/mL | 0.007 | 0.700 | 2.828 | 0.665 |
| 50 mg/mL | 0.361 | 0.375 | 0.262 | 2.645 |

### Example 8

### Enhanced Revascularization of Ischemic Hind Limbs with Cells in a Fibrin Scaffold in Preclinical Studies of Critical Limb Ischemia

On the day of the implant surgery, mice were anesthetized and the surgery was performed under aseptic conditions. Nude mice (8 weeks of age) were subject to iliofemoral artery ligation and excision in one limb, while the second limb was untreated (control). Methods of carrying out hind limb ischemia are described by Rehman et al. (Circulation 109: 1292-1298, 2004). Animals were dosed with cells (adipose-derived cells rich in CD34+ marker) or control (saline) in a fibrin matrix the day following surgery. A small surgical skin incision was made in the groin area. A fibrin gel implants was placed on the caudal side of the hind limb proximal to an arterial blood supply to the hind limb. After surgery, the skin incision was closed and the animals were allowed to recover. Blood flow was quantitatively analyzed by Laser Doppler Imaging (LDI) as an indication of relative reperfusion as taught in Rehman et al. (Circulation 109:1292-98, 2004). LDI provides a non-invasive method for scanning a tissue to examine perfusion. LDI measurements were taken at days 1, 5, 10, 15, and 20. Histology data was also taken for blood vessel measurements.

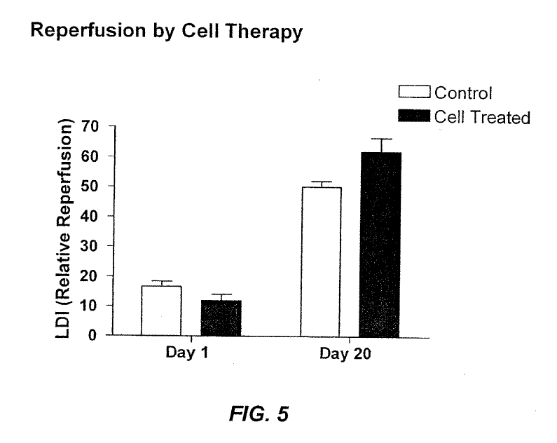

After 20 days, control showed an increase in relative perfusion compared to day 1. See FIG. 5. However, cell-treated demonstrated an even greater increase in relative perfusion than control. Thus, this experiment showed that cell rich in CD34+ enhanced revascularization in a model of hind limb ischemia.

### Example 9

### Crosslinking Occurs in a Fibrin Clot at a Fibrinogen:Thrombin Ratio of 17.5:2

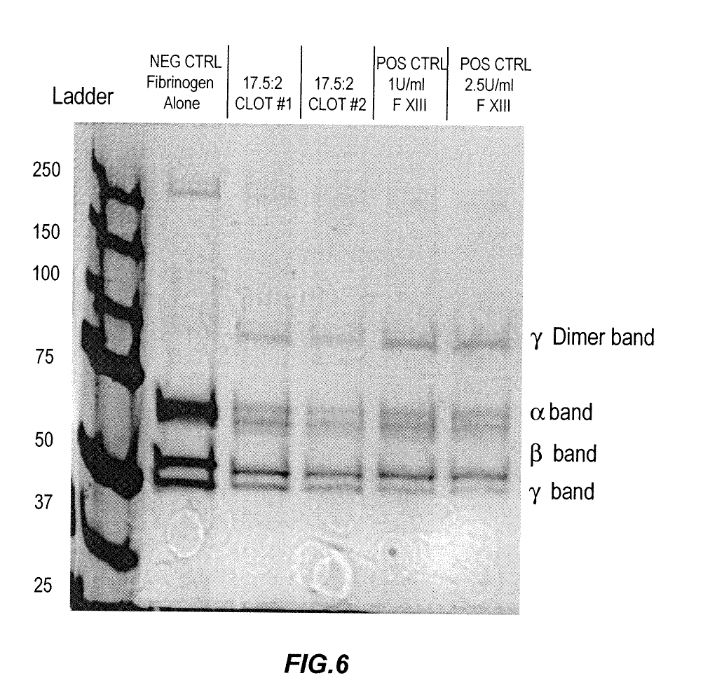

To examine crosslinking in a fibrin clot at the diluted fibrinogen:thrombin concentration ratio of 17.5:2, the following experiment was carried out. Fibrinogen and thrombin solutions were made using Tisseel® kit reagents. Fibrinogen was reconstituted and then diluted to 35 mg/ml using PBS. Thrombin was reconstituted and then diluted to 4 U/ml using PBS. Fibrin clots were formed by adding 50 μL of fibrinogen (diluted) to 50 μL of thrombin (diluted) and mixed for a final concentration of 17.5 mg/ml fibrinogen: 2 U/ml thrombin. Factor XIII was added to the thrombin solutions for positive control (see FIG. 6, lanes 3 and 4) at final concentrations as indicated. Clots were allowed to solidify for 1 hour. After 1 hour, a solution containing 8N urea, 1% SDS, and 1%-β-mercaptoethanol was added; clots were added to a shaker for 10 minutes for degradation and reduction of proteins.

SDS-PAGE gel was run using an equal volume of all clot/protein solutions (see FIG. 6). Fibrinogen control lane is a negative control for the y dimer band which forms as crosslinking in clots increases. (No crosslinking was evident in this lane.) Factor XIII lanes were positive controls as Factor XIII increases crosslinking (as indicated by formation of the y dimer band). Upon comparison of negative and positive controls with clot lanes, it is clear that crosslinking still occurs at the fibrinogen:thrombin concentration ratio of 17.5:2.

The invention has been described in terms of particular embodiments found or proposed to comprise preferred modes for the practice of then invention. It will be appreciated by those of ordinary skill in the art that, in light of the present disclosure, numerous modifications and changes can be made in the particular embodiments exemplified without departing from the intended scope of the invention. Therefore, it is intended that the appended claims cover all such equivalent variations which come within the scope of the invention as claimed.

1. A composition comprising a fibrin clot and stem cells.

2. The composition of claim 1 wherein the stem cells are positive for CD34 (CD34+).

3. The composition of claim 2 wherein the CD34+ cells are isolated by using any CD34+ selection means.

4. The composition of claim 2 wherein the CD34+ cells are present in an amount from about 1,000 to about 10,000,000 cells per 1 mL of fibrin clot.

5. The composition of claim 4 wherein the CD34+ cells are present in an amount from about 25,000 to about 2,000,000 cells per 1 mL of fibrin clot.

6. The composition of claim 5 wherein the CD34+ cells are present in an amount from about 200,00 to about 600,000 cells per 1 mL of fibrin clot.

7. The composition of claim 6 wherein the CD34+ cells are present in an amount of about 300,000 cells per 1 mL of fibrin clot.

8. The composition of claim 1 wherein the fibrin clot is Tisseel® or Tisseel® VHSD.

9. The composition of claim 1 wherein the fibrin clot is in a phosphate buffer or a phosphate-buffered saline solution.

10. The composition of claim 8 wherein the Tisseel® or Tisseel® VHSD is in a phosphate buffer or a phosphate-buffered saline solution.

11. The composition of claim 1 wherein the fibrin clot comprises fibrinogen at a final concentration from about 1 mg/ml to about 100 mg/ml and thrombin at a final concentration from about 1 IU/ml to about 250 IU/ml.

12. The composition of claim 11 wherein the fibrin clot comprises fibrinogen at a final concentration of about 17.5 mg/ml and thrombin at a final concentration of about 2 IU/ml.

13. The composition of claim 11 wherein the fibrinogen and thrombin are in a phosphate buffer or a phosphate-buffered saline solution.

14. A method for treating a localized site of injury or disease in a subject in need thereof, the method comprising the step of delivering a composition comprising a fibrin clot and stem cells to the site of injury or disease in an amount effective for treating the injury or disease.

15. The method of claim 14 wherein the stem cells are positive for CD34 (CD34+).

16. The method of claim 15 wherein the CD34+ cells are isolated by using any CD34+ selection means.

17. The method of claim 15 wherein the CD34+ cells are present in an amount from about 1,000 to about 10,000,000 cells per 1 mL of fibrin clot.

18. The method of claim 15 wherein the CD34+ cells are present in an amount from about 25,000 to about 2,000,000 cells per 1 mL of fibrin clot.

19. The method of claim 15 wherein the CD34+ cells are present in an amount from about 200,00 to about 600,000 cells per 1 mL of fibrin clot.

20. The method of claim 15 wherein the CD34+ cells are present in an amount of about 300,000 cells per 1 mL of fibrin clot.

21. The method of claim 14 wherein the fibrin clot is Tisseel® or Tisseel® VHSD.

22. The method of claim 14 wherein the fibrin clot is in a phosphate buffer or a phosphate-buffered saline solution.

23. The method of claim 21 wherein the Tisseel® or Tisseel®, VHSD is in a phosphate buffer or a phosphate-buffered saline solution.

24. The method of claim 14 wherein the fibrin clot comprises fibrinogen at a final concentration from about 1 mg/ml to about 100 mg/ml and thrombin at a final concentration from about 1 IU/ml to about 250 IU/ml.

25. The method of claim 24 wherein the fibrin clot comprises fibrinogen at a final concentration of about 17.5 mg/ml and thrombin at a final concentration of about 2 IU/ml.

26. The method of claim 24 wherein the fibrinogen and thrombin are in a phosphate buffer or a phosphate-buffered saline solution.

27. A method of enhancing vascularization to a localized site of injury or disease in a subject in need thereof, the method comprising the step of delivering a composition comprising a fibrin clot and stem cells to the site of injury or disease in an amount effective for enhancing vascularization.

28. The method of claim 27 wherein the stem cells are positive for CD34 (CD34+).

29. The method of claim 28 wherein the CD34+ cells are isolated by using any CD34+ selection means.

30. The method of claim 28 wherein the CD34+ cells are present in an amount from about 1,000 to about 10,000,000 cells per 1 mL of fibrin clot.

31. The method of claim 28 wherein the CD34+ cells are present in an amount from about 25,000 to about 2,000,000 cells per 1 mL of fibrin clot.

32. The method of claim 28 wherein the CD34+ cells are present in an amount from about 200,00 to about 600,000 cells per 1 mL of fibrin clot.

33. The method of claim 28 wherein the CD34+ cells are present in an amount of about 300,000 cells per 1 mL of fibrin clot.

34. The method of claim 27 wherein the fibrin clot is Tisseel® or Tisseel® VHSD.

35. The method of claim 27 wherein the fibrin clot is in a phosphate buffer or a phosphate-buffered saline solution.

36. The method of claim 34 wherein the Tisseel® or Tisseel® VHSD is in a phosphate buffer or a phosphate-buffered saline solution.

37. The method of claim 27 wherein the fibrin clot comprises fibrinogen at a final concentration from about 1 mg/ml to about 100 mg/ml and thrombin at a final concentration from about 1 IU/ml to about 250 IU/ml.

38. The method of claim 37 wherein the fibrin clot comprises fibrinogen at a final concentration of about 17.5 mg/ml and thrombin at a final concentration of about 2 IU/ml.

39. The method of claim 37 wherein the fibrinogen and thrombin are in a phosphate buffer or a phosphate-buffered saline solution.

40. A method of treating ischemia in a subject, comprising the step of delivering a composition comprising a fibrin matrix and stem cells to a site of ischemia in an amount effective to treat ischemia.

41. The method of claim 40 wherein the stem cells are positive for CD34 (CD34+).

42. The method of claim 41 wherein the CD34+ cells are isolated by using any CD34+ selection means.

43. The method of claim 41 wherein the CD34+ cells are present in an amount from about 1,000 to about 10,000,000 cells per 1 mL of fibrin clot.

44. The method of claim 41 wherein the CD34+ cells are present in an amount from about 25,000 to about 2,000,000 cells per 1 mL of fibrin clot.

45. The method of claim 41 wherein the CD34+ cells are present in an amount from about 200,00 to about 600,000 cells per 1 mL of fibrin clot.

46. The method of claim 41 wherein the CD34+ cells are present in an amount of about 300,000 cells per 1 mL of fibrin clot.

47. The method of claim 40 wherein the fibrin clot is Tisseel® or Tisseel® VHSD.

48. The method of claim 40 wherein the fibrin clot is in a phosphate buffer or a phosphate-buffered saline solution.

49. The method of claim 47 wherein the Tisseel® or Tisseel® VHSD is in a phosphate buffer or a phosphate-buffered saline solution.

50. The method of claim 40 wherein the fibrin clot comprises fibrinogen at a final concentration from about 1 mg/ml to about 100 mg/ml and thrombin at a final concentration from about 1 IU/ml to about 250 IU/ml.

51. The method of claim 50 wherein the fibrin clot comprises fibrinogen at a final concentration of about 17.5 mg/ml and thrombin at a final concentration of about 2 IU/ml.

52. The method of claim 50 wherein the fibrinogen and thrombin are in a phosphate buffer or a phosphate-buffered saline solution.

53. A kit for preparing a fibrin matrix comprising stem cells, the kit comprising:
(a) a first vial or first storage container comprising fibrinogen;
(b) a second vial or second storage container comprising thrombin; and
(c) a third vial or third storage container comprising stem cells, said kit further optionally containing a phosphate buffer and instructions for use thereof.

54-68. (canceled)
